(19) United States
(12) Patent Application Publication
(10) Pub. No.: US 2013/0121916 A1
(43) Pub. Date: May 16, 2013

(54) Compositions for Colon Lavage and Methods of Making and Using Same

(71) Applicant: Sergio Baroni, Villa D'adda (IT)

(72) Inventor: Sergio Baroni, Villa D'adda (IT)

(21) Appl. No.: 13/583,349

(22) PCT Filed: Mar. 10, 2011
(86) PCT No.: PCT/EP11/01183

§ 371 Date: Nov. 8, 2012

Related U.S. Application Data
(60) Provisional application No. 61/356,111, filed on Jun. 18, 2010.

(30) Foreign Application Priority Data
Mar. 10, 2010 (EP) 10425061.8

(51) Int. Cl. A61K 49/00

(52) U.S. Cl. CPC A61K 49/006
USPC 424/9.1

(57) ABSTRACT

Disclosed herein are compositions that include polyethylene glycol; alkali metal sulfate; electrolytes selected from the group consisting of sodium bicarbonate, sodium chloride, and potassium chloride or a mixture thereof; and a gastro-protected dye composition comprising a gastro-protectant and a dye suitable for use in an internal colon examination procedure. Also provided herein are sachets, and containers, e.g. sachets that include disclosed compositions; kits for colon cleansing, and aqueous solutions suitable for colon cleansing.

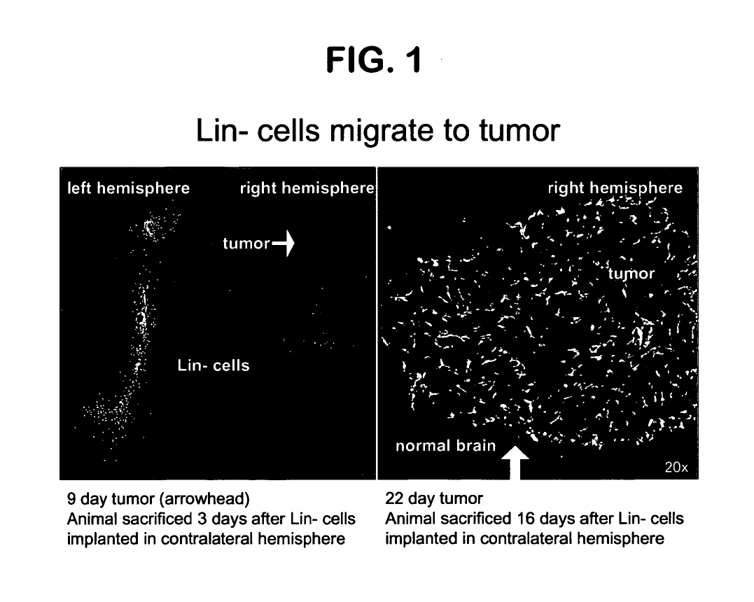

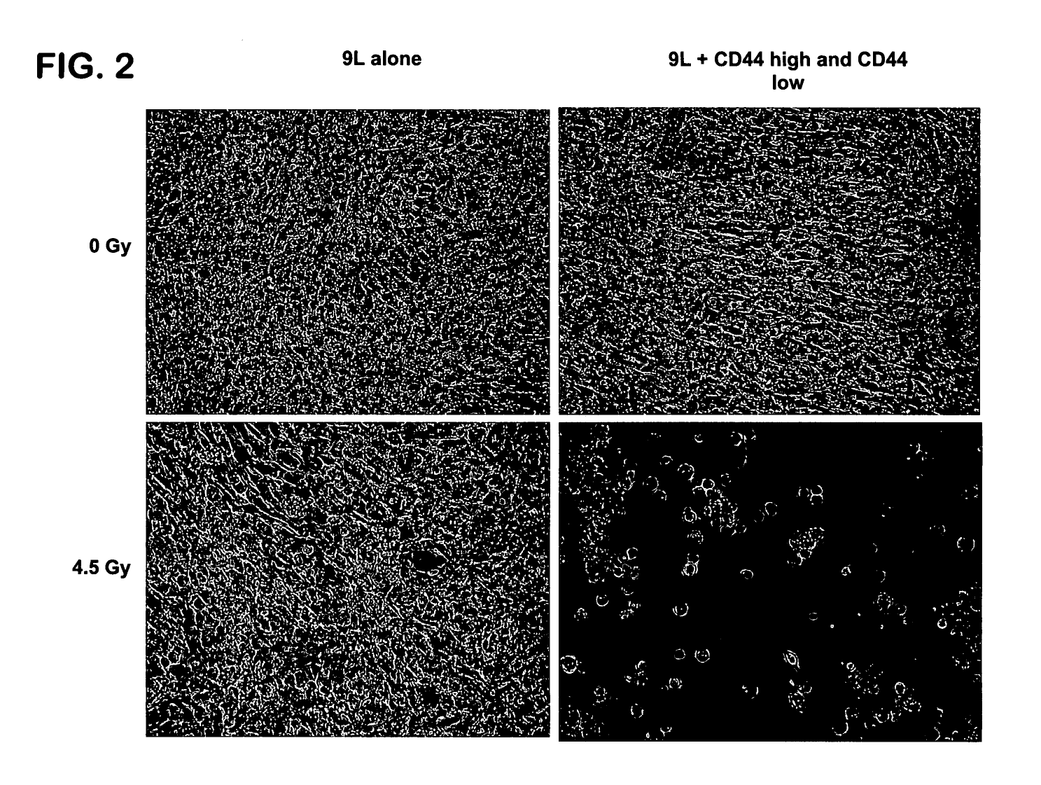

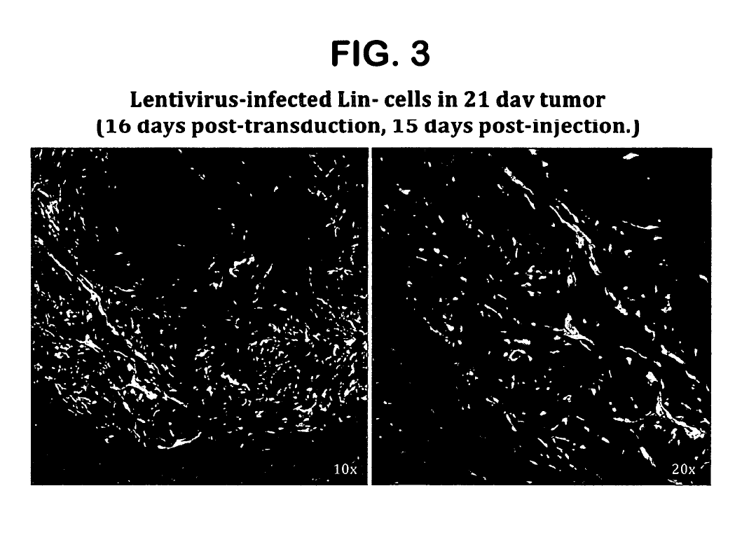

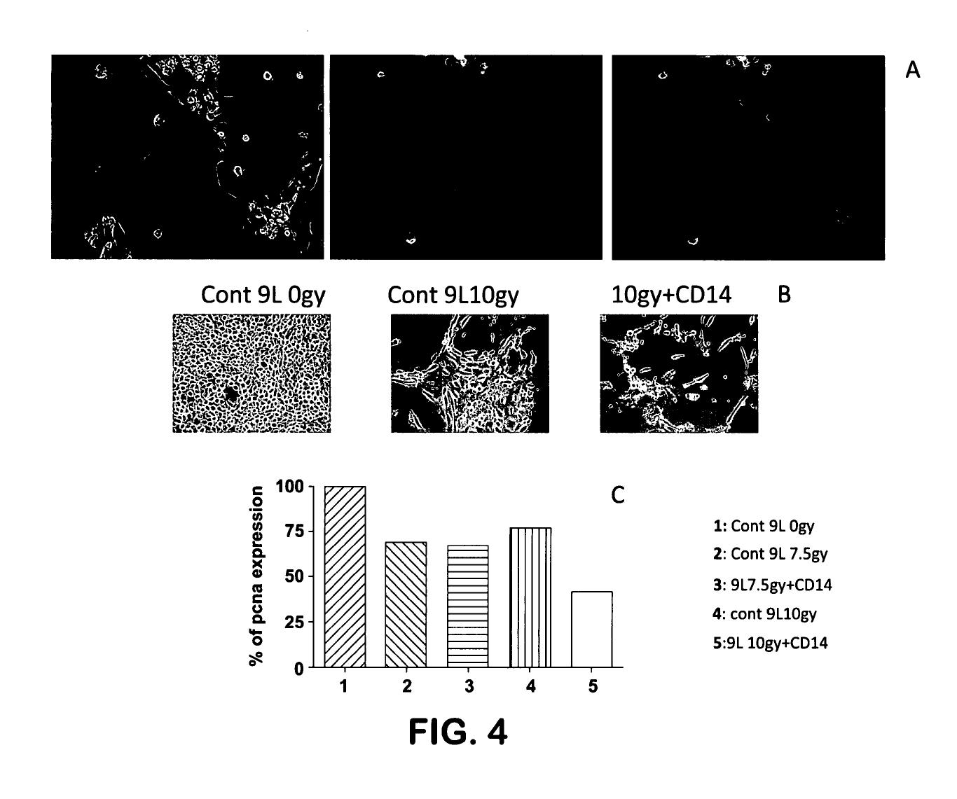

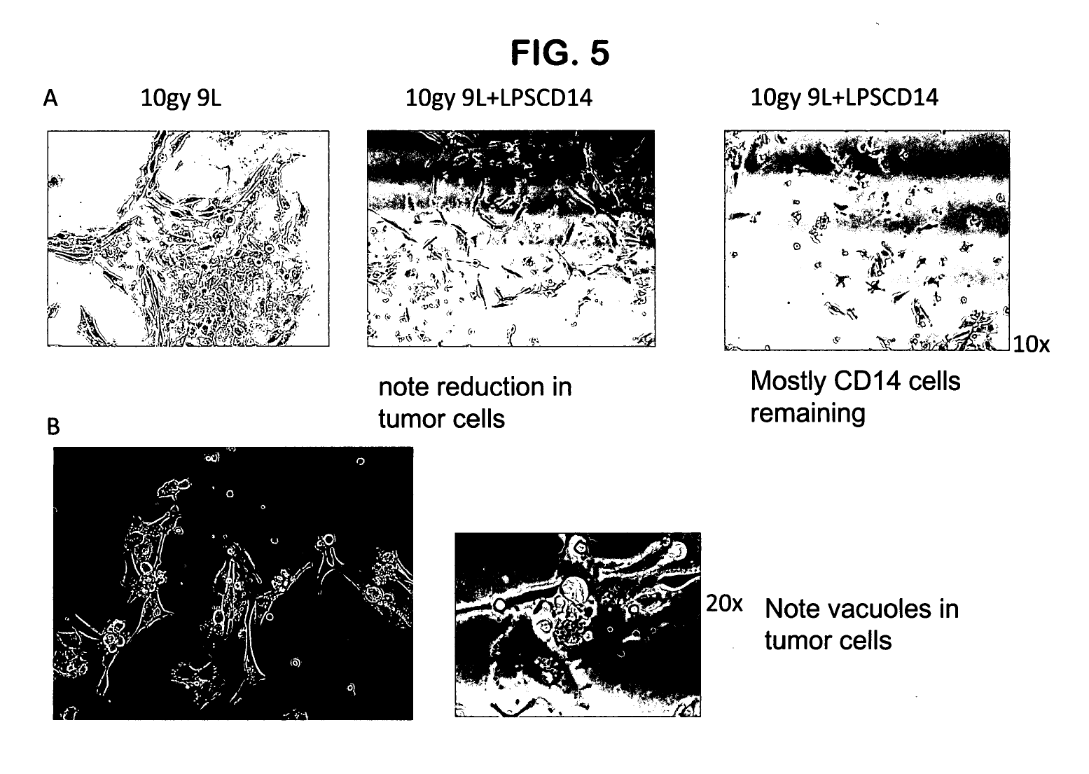

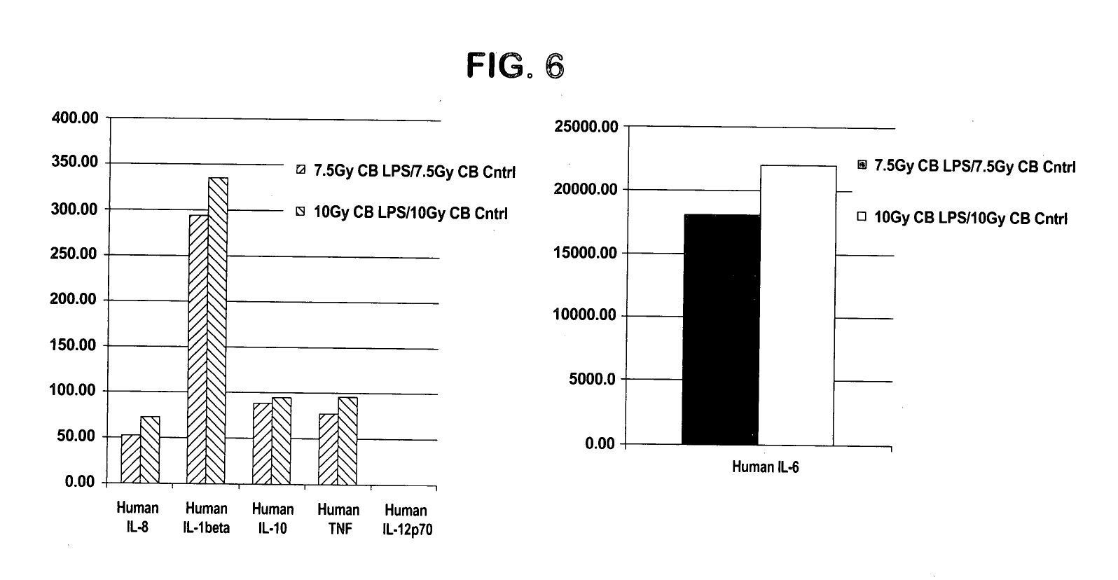

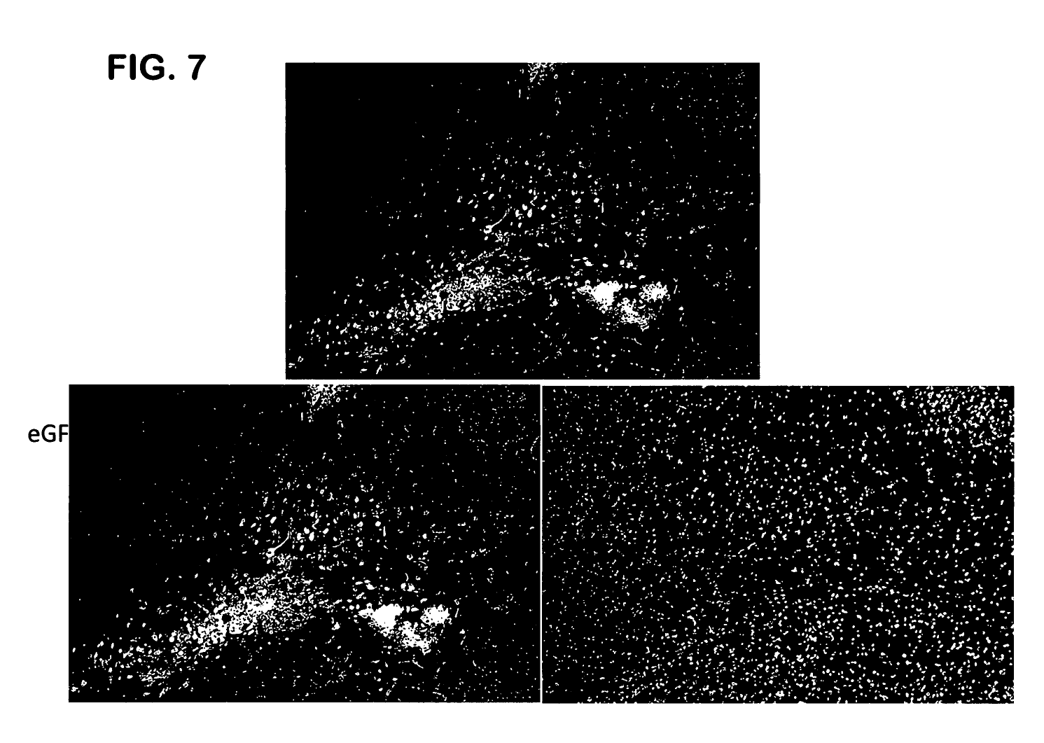

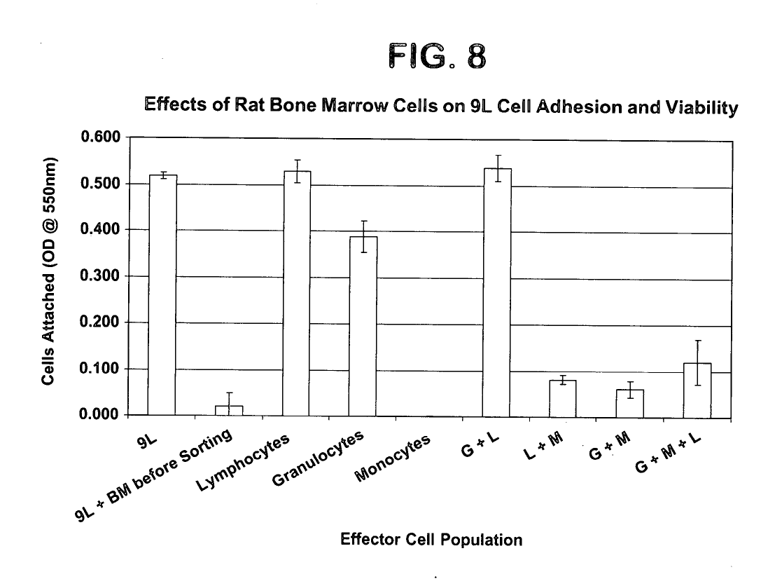

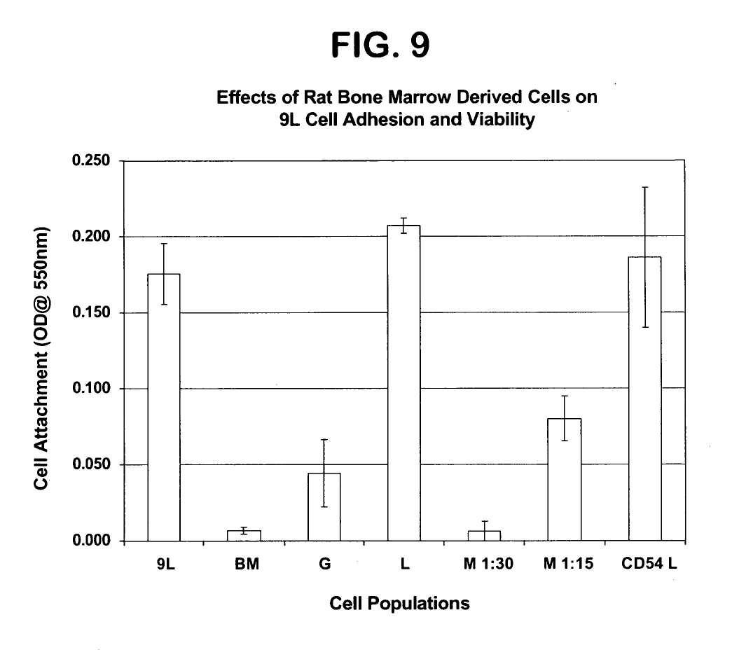

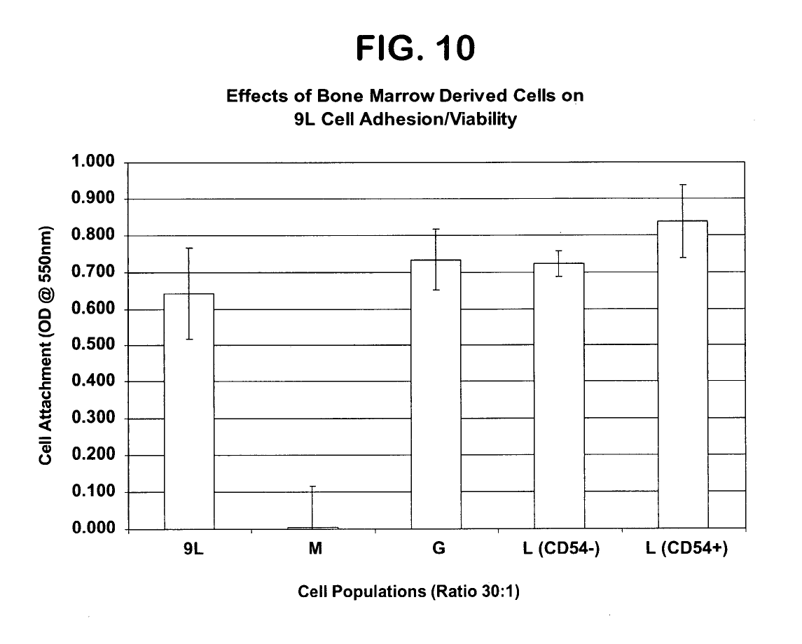

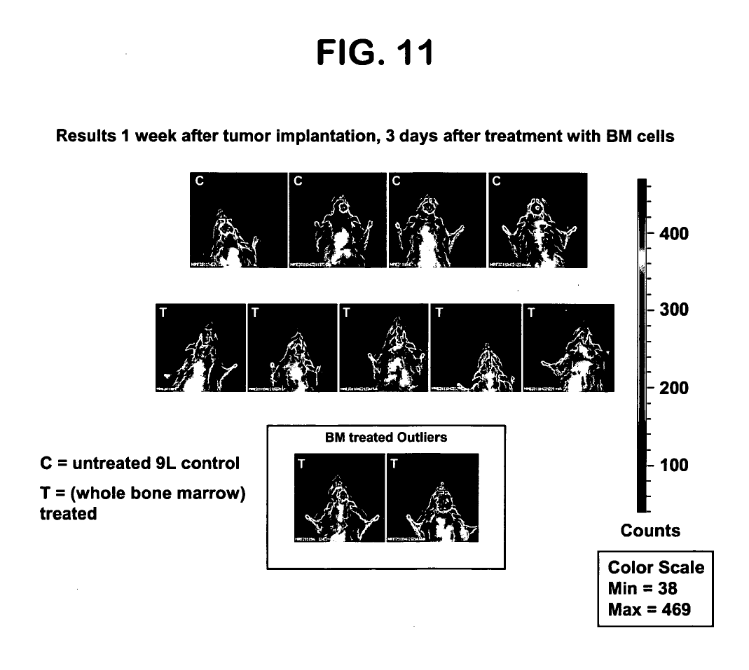

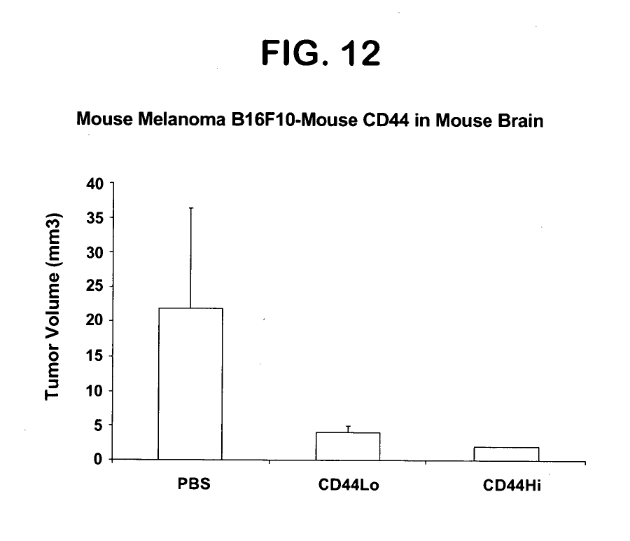

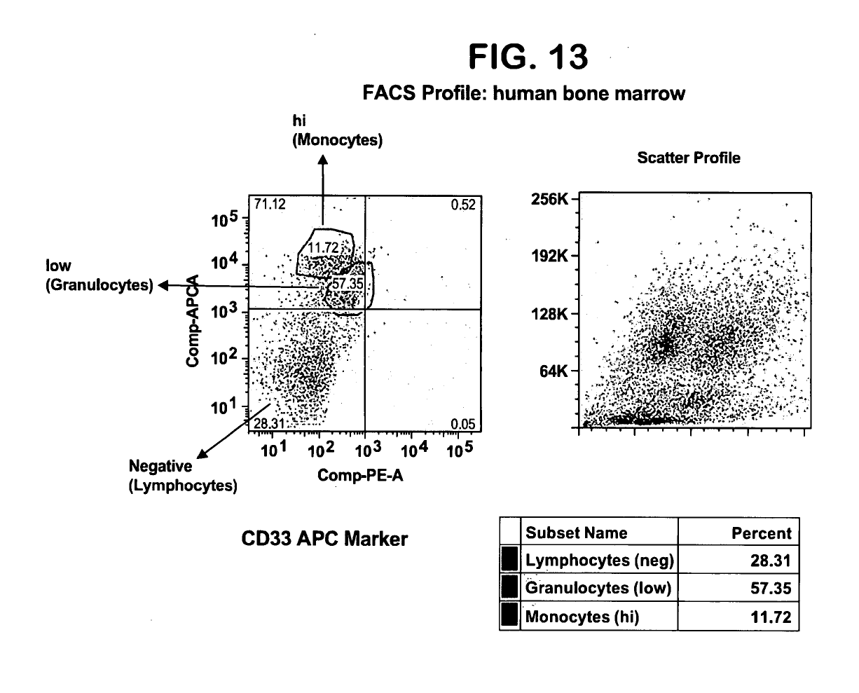

### RELATED APPLICATIONS

This application claims priority to EP10425061.8, filed Mar. 10, 2010, and U.S. Ser. No. 61/356,111, filed Jun. 18, 2010, both of which are incorporated by reference in their entirety.

### BACKGROUND

Patients who are undergoing surgical procedures or diagnostic examinations of the large bowel usually undergo preparation to assure that the bowel is cleansed of all fecal material adequately before the procedure. This serves to minimize contaminating the operating area for example, during surgery for explorations of potential masses or for bowel resection. An additional purpose is to allow a clean interior surface of the colon for diagnostic examination, for example during endoscopic surveillance as a diagnostic examination for detecting colon cancer.

For example, sigmoidoscopy, colonoscopy, radiographic examination, preparation for patients undergoing bowel surgery, and other medical or diagnostic procedures on the bowels or colon, all require that the bowels and colon be thoroughly purged and cleaned for accurate examination. In particular, it is essential that as much fecal matter as possible be removed from the colon to permit adequate visualization of the intestinal mucosa. This is important prior to, for example, diagnostic procedures such as flexible sigmoidoscopy or colonoscopy, diagnostic examinations widely performed to screen patients for diseases of the colon. In addition, it is important that the intestines be cleansed thoroughly in order to obtain satisfactory radiographs of the colon.

In order to more accurately visualize lesions, polyps and other abnormal growths during e.g., a colonoscopy, however, further spraying of dye may be performed during a colon procedure, e.g., chromoendoscopy. In that procedure the lining of the colon is sprayed with a dye, typically with a catheter, and the dye is taken up by abnormalities such as pre-lesions and polyps, thereby making such abnormalities much more visible and allowing easier identification and a better diagnosis. Studies in which the patients have routine colonoscopy and chromoendoscopy performed the same day demonstrate that more polyps are found with chromoendoscopy. However, such chromoendoscopies are not widely used in part because the procedure is messy and because it takes more time to complete the colonoscopy. Further, images may vary depending on how the dye is topically applied.

There is therefore a need for more convenient and effective methods and compositions that can assist in reducing the duration of a chrom-endoscopic procedure and also allowing easier identification of e.g. pre-lesions.

### SUMMARY

This disclosure provides generally compositions, kits and methods that are capable of allowing dyeing or staining of the colon to assist in a diagnostic or other procedure, while also achieving the cleansing of the colon also necessary for such a procedure.

For example, provided herein is a dry composition comprising polyethylene glycol; an alkali metal sulfate; electrolytes selected from the group consisting of sodium bicarbonate, sodium chloride, and potassium chloride or a mixture thereof; and a gastro-protectant dye composition suitable for use in an internal colon examination procedure comprising a gastro-protectant and a dye. Contemplated gastro-protectant dye compositions may include e.g. dye microencapsulated by the gastroprotectant or dye particles film coated by the gastroprotectant. In an embodiment, gastro-protected dye compositions that form part of disclosed dry compositions may be capable of releasing the dye at a pH of greater than about 5.

Also contemplated herein is a container, e.g., a sachet, a packet, or an envelope containing disclosed dry compositions. Such a container may include an amount of dry composition in the container that is sufficient to prepare 2 to 4 liters of a colon cleansing solution.

Provided herein is a kit for a colonoscopy preparatory solution comprising: a) a first container containing a first dry composition comprising: (i) 25 g to 125 g of polyethylene glycol; (ii) 0.5 g to 4 g of an alkali metal sulfate; and (iii) 0.3 g to 2 g of electrolytes selected from the group consisting of sodium bicarbonate, sodium chloride, and potassium chloride or a mixture thereof; and b) a gastro-protected dye composition comprising a gastro-protectant and a dye suitable for use in an internal colon examination procedure, and optionally instructions for use.

In an embodiment, a method of staining the colon of a mammal for diagnostic study while cleansing the colon of a mammal is provided, that comprises orally administering about 1.5 to 4 liters of a solution that comprises about 50 g/L to about 360 g/L polyethylene glycol to the mammal; and orally administering a gastro-protected dye composition comprising a gastro-protectant and a dye suitable for use in an internal colon examination procedure, to the mammal. For example, the method may include substantially simultaneous oral administration of the solution and the gastro-protected dye composition.

### DETAILED DESCRIPTION

The present disclosure provides herein, at least in part, a dry composition, that when mixed with water, provides a colon cleansing and staining action in the colon that is effective when administered to a patient in need thereof, e.g. in a 2L quantity, a 3L quantity and/or a 4L quantity.

In some embodiments, the disclosure provides for a dry composition comprising polyethylene glycol (e.g., having an average molecular weight of 3300 or 4000 g/mol, for example, a PEG 3350); optionally an alkali metal sulfate; and optionally one or more electrolytes selected from the group consisting of sodium bicarbonate, sodium chloride, and potassium chloride or a mixture thereof; and a gastro-protectant dye composition suitable for use in an internal colon examination procedure comprising a gastro-protectant and a dye suitable for colon staining or coloring. For example, contemplated gastro-protectant dye compositions may include a dye microencapsulated by a gastroprotectant material, and/or may include a dye (e.g. dye particles formed by granulation, film coated by a gastroprotectant material (e.g., a material such as an enteric material that may not dissolve, upon oral administration, until the material reaches the small intestine.) Such contemplated gastro-protected dye compositions may release a dye at a pH of greater than about 5.

Such gastro-protected dye compositions may be formed by those skilled in the art. For example, such compositions may be achieved by e.g. granulating a dye compound and film coating the granulated particles with a disclosed gastroprotective material. Alternatively, dye particles may be microencapsulated as known to those of skill in the art e.g., by coacervation, spray-drying or fluid-bed granulation.

Contemplated dyes which may be used in the compositions disclosed herein may include one or more of the following: Curcumin (i) Riboflavin (ii) Riboflavin-5′-phosphate, Tartrazine, Quinoline Yellow, Sunset Yellow, FCF OrangE, Yellow S, Cochineal, Carminic acid, Carmines, Azorubine, Carmoisine, Ponceau 4R, Cochineal Red A, Allura Red AC, Patent Blu EV, Indigotine, Indigo carmine, Brilliant Blue FCF, Chlorophylls and chlorophyllins, Copper complexes of chlorophylls and chlorophyllins, Green S, Plain caramel, Brilliant Black BN, Black PN, Vegetable carbon, Brown HT, Carotenes, Lutein, Beetroot Red, betanin, Anthocyanins, Calcium carbonate, Titanium dioxide, Iron oxides and hydroxides, Amaranth, Brown FK, Erythrosine, Lithol Rubine BK, Red 2G.

Other contemplated dyes include Acid fuchsine, Alba red, Alizarin cyanine green F, Alizurol purple S5, Allura Red AC, Alphazurine FGBrilliant lake red R, Dibromofluorescein, Diiodofluorescein, Eosine, Erythrosine yellowish Na, Fast green FCF, Flaming red, Fluorescein, Helindone pink CN, Indanthrene blue, Lake bordeaux B, Lithol rubin B Ca, Naphthol yellow 5, Orange Il, Phloxine B, Ponceau 5X, Pyranine concentrated, Quinizarinegreen 5S, Tetrabromofluorescein, Tetrachlorotetrabromofluorescein, Toney red, and Uranine.

Contemplated dyes may include Alcian Blue, Anazolene Sodium, Brilliant Green, Cantaxanthin, Carthamin, Citrus Red 2, Evan's Blue, Fast Green FCF, Indocyanine Green, Methyl Blue, Methylene Blue, N-(p-Methoxyphenyl)-p-phenylenediamine, Ponceau 3R, Ponceau SX, Pyranine, Rhodamine B, Saunders Red, Sudan Black B, Sulphan Blue, Tolonium Chloride, and/or Vital Red.

Examples of such gastroprotectant materials that may be suitable for use in the disclosed compositions include one or more of the following: methacrylic acid-methylmethacrylate copolymers such as: methacrylic acid-methylmethacrylate copolymer 1:1 (type A), methacrylic acid-methylmethacrylate copolymer 1:2 (type B),methacrylic acid-ethylacrylate copolymers, and/or mixtures thereof; shellac, cellulose derivatives such as: cellulose acetate phthalate, cellulose acetate succinate, hydroxypropyl methyl cellulose phthalate, hydroxy propyl methyl cellulose acetate succinate (hypromellose acetate succinate), polyvinyl acetate phthalate (PVAP), sodium alginate, stearic acid, natural waxes such as: beeswax, carnauba wax, synthetic waxes such as polyethyleneglycol adipate, cetostearyl alcohol, and/or cetyl palmitate.

For example, a contemplated gastroprotectant component may be selected from the group consisting of: methacrylic acid-methylmethacrylate copolymer, methacrylic acid-ethylacrylate copolymers, shellac, cellulose acetate phthalate, cellulose acetate succinate, hydroxypropyl methyl cellulose phthalate, hydroxy propyl methyl cellulose acetate succinate, polyvinyl acetate phthalate, sodium alginate, beeswax, carnauba wax, polyethyleneglycol adipate, cetostearyl alcohol or cetyl palmitate. In a particular embodiment, a contemplated gastro-protected dye composition may include methacrylate or a copolymer thereof.

One or more alkali or alkaline metal sulfates may be included in the disclosed compositions, for example, sodium sulfate. One or more electrolytes may optionally or additionally form part of a disclosed dry composition, e.g., may be selected from the group consisting of sodium bicarbonate, sodium chloride, and potassium chloride or a mixture thereof;

In some embodiments, a flavorant may be present to add or modify a flavor in the disclosed compositions. Examples of flavorants include anise oil, cinnamon oil, vanilla, vanillin, cocoa, chocolate, menthol, grape, peppermint oil, oil of wintergreen, clove oil, bay oil, eucalyptus, thyme oil, cedar leaf oil, oil of nutmeg, oil of sage, oil of bitter almonds, cassia oil, citrus oils such as lemon, orange, lime and grapefruit oils, and fruit essences, including apple, pear, peach, berry, wildberry, date, blueberry, kiwi, strawberry, raspberry, cherry, plum, pineapple, and apricot. Sweeteners may be included such as sugar, acesulfame K, sodium cyclamate, saccharin, sucrolose, and/or mixtures thereof.

A preservative may be optionally present to provide a longer shelf life to a pre-packaged composition. Examples of preservatives include potassium sorbate and sodium benzoate.

An excipient may be present to provide stability and/or flowability to the ingredients in the composition. Examples of excipients include silica, cellulose, cellulose esters, and lecithin. Examples of excipients affecting viscosity may also include the gelling agents when present in low concentrations.

A disclosed composition, or distinct parts of a disclosed composition, may be packaged in a container, such as a sachet, a packet, an envelope, a tube, an ampoule, a bottle, or a tub. The amount of a composition in an individual container may be sufficient to prepare a dose suitable for one colon procedure when mixed with water. For example, the amount of composition may provide 2, 3, or 4 L of an aqueous solution to be delivered orally to a patient in need of colon preparation.

In an embodiment, a colon cleansing and staining aqueous solution is provided that includes a disclosed composition and about 2 to about 4 liters of water. For example, 1 liter of a disclosed aqueous solution may have an osmolarity of 235 to 304 mOsmol/kg. In another embodiment, 1 liter of a disclosed aqueous solution may have an osmolarity of 330 to 550 mOsmol/kg.

Alternatively, a disclosed dry or aqueous composition may be packaged in a container such as a glass or plastic bottle, a plastic pouch, or a paper-based carton. In one example, a disclosed aqueous solution may be formed by combining water with one or more ingredients of a disclosed composition, agitating and/or heating the mixture to dissolve the ingredients, and then packaging the solution in a container.

For example, contemplated herein is a container (e.g., a sachet, a packet, or an envelope) containing a disclosed dry composition. Such a container may include the amount of dry composition sufficient to prepare 2 to 4 liters of a colon cleansing solution. For example, a contemplated container may include a disclosed dry composition that includes about 28 g to about 31 g of polyethylene glycol, about 56 to 62 g/L polyethylene glycol, or about 90 to 150 g/L polyethylene glycol. In an embodiment, a disclosed container may include (a) 25 g to 125 g of polyethylene glycol; (b) 0.5 g to 4 g of an alkali metal sulfate; and (c) 0.3 g to 2 g of the electrolytes, and dye, e.g. a gastroprotected dye composition.

Contemplated containers may include a laxative, e.g. bisacodyl, sodium picosulfate, dantrone, bisoxatin, cascara, or senna-based laxative (e.g., senna), and/or ascorbic acid or salts thereof. Alternatively, a disclosed dry composition may be provided in a unit form, e.g. in a sachet, or in two or more component form, in which some ingredients or parts of the composition (e.g. absorbic acid) are packaged separately from the other components.

For example, provided herein is a dry composition, that when dissolved in e.g. 2 or 4 liters of water provides an oral solution that is iso-osmolar to body fluids. Such a dry composition may include polyethylene glycol 4000 or polyethylene glycol 3350, sodium bicarbonate, sodium chloride, sodium sulfate (anhydrous), and potassium chloride, and may not contain not less than 90.0 percent and not more than 110.0 percent of the labeled amounts of polyethylene glycol 3350, potassium (K⁺), sodium (Na⁺), bicarbonate (HCO₃), chloride (Cl⁻), and sulfate (SO₄⁻). In this exemplary embodiment, when added to water, the T iso-osmolar concentrations solutions are: potassium: 10 mEq; sodium: 125 mEq; bicarbonate: 20 mEq; chloride: 35 mEq; sulfate: 80 mEq equivalent to 40 mmol, and dye. In some embodiments, the dye should not substantially interfere with the osmolarity of the final solution, and for example, osmolarity may be within the range 235-304 mOsm e.g., as tested by USP <785>.

Also provided herein is a kit for a colonoscopy preparatory solution that includes a) a first container containing a first dry composition, e.g. a disclosed dry composition, for example, that includes: (i) 25 g to 125 g of polyethylene glycol (e.g. about 28 g to about 31 g of polyethylene glycol); (ii) 0.5 g to 4 g of an alkali metal sulfate (e.g. sodium sulfate) ; and (iii) 0.3 g to 2 g of electrolytes selected from the group consisting of sodium bicarbonate, sodium chloride, and potassium chloride or a mixture thereof; and b) a dye suitable for use in an internal colon examination procedure, e.g. a gastro-protected dye composition comprising a gastro-protectant and such a dye, (e.g. about 1 g to about 10 g gastro-protected dye composition), and optionally instructions for use.

In some embodiments, the first container (e.g. a sachet) contains the dye or the gastro-protected dye composition. In another embodiment, a contemplated kit further comprising a second container (e.g. a sachet or a 1 to 5 liter container suitable for mixing a dry composition with water) that contains the gastro-protected dye composition. Contemplated kits may include a laxative (e.g., in tablet form), or an additional container e.g., comprising ascorbic acid or the laxative. Contemplated laxatives include bisacodyl, sodium picosulfate, and a senna-based laxative, e.g. senna.

For example, a kit for a colon preparatory solution is provided comprising a container that contains a dry composition, wherein the dry composition comprises: (a) one or more electrolytes each selected from the group consisting of sodium sulfate, sodium phosphate, sodium picosulfate, or magnesium citrate; and b) a gastro-protected dye composition comprising a gastro-protectant and a dye suitable for use in an internal colon examination procedure.

Also provided herein are methods of staining the colon of a mammal for diagnostic study while cleansing the colon of a mammal, comprising: orally administering about 1.5 to 4 liters of a solution that comprises about 60 g/L to about 360 g/L polyethylene glycol to the mammal; and orally administering a gastro-protected dye composition comprising a gastro-protectant and a dye suitable for use in an internal colon examination procedure, to the mammal. Such oral administration of the solution and orally administering the gastro-protected dye composition may be substantially simultaneous. Contemplated methods may further comprise administering a stimulant laxative before orally administering the solution and/or the dye composition.

For example, administering an effective amount of a disclosed aqueous solution, e.g. in combination with administering a laxative, may induce colonic purgation while staining the colon in preparation for e.g. a colon examination. In an embodiment, a laxative is administered and allowed to produce a bowel movement, followed by administering an amount of a disclosed aqueous solution.

### EXAMPLES

The compositions disclosed herein can be prepared in a number of ways well known to one skilled in the art, for example, compositions may be prepared using the reactions and techniques described herein. In the description of the methods described below, it is to be understood that all proposed reaction conditions, including choice of solvent, reaction atmosphere, reaction temperature, duration of the experiment and workup procedures, can be chosen to be the conditions standard, unless otherwise indicated. The starting materials for the examples are either commercially available or are readily prepared by standard methods from known materials.

### Example 1

### Mixture for Oral Solution Containing Methylene Blue

### Enteric Coating of Methylene Blue

A solution was prepared by mixing 1.5 kg methylmethacrylate copolymer type A, 0.15 kg of triethylcitrate and 20 kg of ethanol 99%.

Under continuous stirring, 1.5 kg of methylene blue was added. Methylene blue is sparingly soluble in ethanol and in some embodiments, finely dispersed in the solution.

This mixture was transferred at the rate of 50 L/h to a spray drying apparatus (Niro atomizer) with the air pressure at the turbine adjusted to maintain a constant spinning rate of 30000 rpm. The temperature was adjusted until a fine powder was obtained at the discharge vent. The drying process was continued until all the material was recovered.

The almost spherical particles obtained were covered with the methacrylate copolymer and released the inner dye only at pH>5.

### Preparation of Mixture

The following ingredients were combined in a suitable stainless steel mixer:

| | | |
|---|---|---|
| Sodium sulfate | 2.843 | kg |
| Sodium bicarbonate | 0.843 | kg |
| Sodium chloride | 0.733 | kg |
| Potassium chloride | 0.371 | kg |

The ingredients were mixed thoroughly for 15 minutes. Then, the following ingredients were added to the mixer:

| | | |
|---|---|---|
| Polyethyleneglycol 4000 | 29.5 | kg |
| Acesulfame K | 0.078 | kg |
| Sodium cyclamate | 0.096 | kg |
| Saccarin acid | 0.021 | kg |
| Natural flavor | 0.315 | kg |

Finally, enteric coated methylene blue equivalent to 1 kg of dye was added. The components were mixed for 15 minutes until the color appeared uniformly dispersed.

### Example 2

### Mixture I for Oral Solution Containing Gastro-Protected Methylene Blue

### Enteric Coating of Methylene Blue

A solution was prepared by mixing 10 kg methylmethacrylate copolymer type A, 1.2 kg of triethylcitrate and 120 kg of absolute ethanol. The mixer was connected to a peristaltic pump and to a fluid bed dryer equipped with a Wurster apparatus for coating. 15 kg of powdered methylene blue was loaded into the fluid-bed apparatus (Glatt or equivalent) and coated by spraying the already prepared mixture at a rate of approximately 1.2-1.7 L/min. At the end of the spraying process, the process was continued in the fluid-bed for about 10-20 minutes to completely dry the mixture. The enterically coated methylene blue was then discharged from the fluid-bed apparatus.

### Preparation of Mixture

The following ingredients were combined in a suitable mixer:

| | | |
|---|---|---|
| Sodium sulfate | 5.5 | kg |
| Sodium bicarbonate | 1.6 | kg |
| Sodium chloride | 1.4 | kg |
| Potassium chloride | 0.71 | kg |

The ingredients were mixed for 15-20 minutes. Then, the following ingredients were added to the mixer:

| | | |
|---|---|---|
| Polyethyleneglycol 4000 | 57 | kg |
| Acesulfame K | 0.15 | kg |
| Sodium cyclamate | 0.18 | kg |
| Saccarin acid | 0.04 | kg |
| Natural flavor | 0.63 | kg |
| Enterically coated methylene blue | 3.49 | kg |

The components were mixed for 15 minutes or until the color appeared uniformly distributed in the powder mass.

### Example 3

### Mixture II for Oral Solution Containing Gastro-Protected Methylene Blue

### Preparation of Mixture

The following ingredients are combined in a suitable mixer:

| | | |
|---|---|---|
| Sodium sulfate | 5.5 | kg |
| Sodium bicarbonate | 1.6 | kg |
| Sodium chloride | 1.4 | kg |
| Potassium chloride | 0.71 | kg |

The ingredients are mixed for 15-20 minutes. Enterically coated methylene blue is prepared as in Example 2. Then, the following ingredients are added to the mixer:

| | | |
|---|---|---|
| Polyethyleneglycol 3350 | 57 | kg |
| Acesulfame K | 0.15 | kg |
| Sodium cyclamate | 0.18 | kg |
| Saccarin acid | 0.04 | kg |
| Natural flavor | 0.63 | kg |
| Enterically coated methylene blue | 3.49 | kg |

The components are mixed for 15 minutes or until the color appeared uniformly distributed in the powder mass.

### Example 4

### Mixture for Oral Solution Containing Coated Indigo

### Preparation of Solution

A solution is prepared by mixing 10 g methylmethacrylate copolymer type C, 1 g of triethylcitrate and 1 L of pure ethanol. Under continuous stirring 20 g of Indigo carmine is added. The obtained mixture is fed to a spray dryer apparatus (Buchi AG or equivalent) at a rate of 5-25 mL/min with the air pressure of the turbine adjusted to maintain a constant spinning rate of 12000 rpm, and the temperature is adjusted until a fine powder is obtained at the discharge vent and the drying process is continued until all the material has been recovered.

The particles obtained release the coloring agent at pH higher than 5-5.5, therefore preventing the staining of the oropharyngeal and the upper digestive tract and allowing the staining to begin in the intestine.

### Preparation of Mixture

The following salts are passed through a 100 mesh screen and are placed in the jar of a turbula mixer of equivalent apparatus:

| | | |
|---|---|---|
| Sodium sulfate | 5.41 | g |
| Sodium bicarbonate | 1.60 | g |
| Sodium chloride | 1.39 | g |
| Potassium chloride | 0.705 | g |

The mixture is mixed well. The following ingredients are added to the mixer:

| | | |
|---|---|---|
| Polyethyleneglycol 4000 | 56.1 | g |
| Sodium cyclamate | 0.183 | g |
| Saccarin acid | 0.0399 | g |
| Natural flavour | 0.630 | g |

The enteric coated indigo carmine is added (3.1 g (equivalent to 2 g of dye), and the mixture is mixed for 15 minutes until the color appears uniformly dispersed.

### Example 5

### Mixture with Gastroprotected Erythrosine

### Coating of the Coloring Agent

A solution mixing 1 kg methylmethacrylate copolymer type B, 0.12 kg of triethylcitrate and 10 L of absolute ethanol is prepared. The mixer is connected to a fluid bed dryer through peristaltic pump, and the fluid bed is equipped with the Wurster cone for coating particles. 1.5 kg of erythrosine (E 127) is loaded into the fluid-bed apparatus (Glatt or equivalent) and coated by spraying the already prepared mixture at a rate of approximately 0.8-1.5 L/min. At the end of the spraying process the process is continued in the fluid-bed for about 10-20 minutes to completely dry the mixture.

### Preparation of Bulk Finished Product

The sodium sulfate, sodium bicarbonate, sodium chloride and potassium chloride is milled in order to reduce their particle size to around 100 mesh. Then, in a suitable mixer, the following is added and mixed for 15-20 minutes:

| | | |
|---|---|---|
| Sodium sulfate | 5.57 | kg |
| Sodium bicarbonate | 1.65 | kg |
| Sodium chloride | 1.44 | kg |
| Potassium chloride | 0.727 | kg |

Then the following is added to the mixer and mix for 15 minutes or until obtaining a uniformly distributed powder mass:

| | | |
|---|---|---|
| Polyethyleneglycol 4000 | 57.8 | kg |
| Acesulfame K | 0.153 | kg |
| Sodium cyclamate | 0.188 | kg |
| Saccarin acid | 0.0412 | kg |
| Natural flavour | 0.5 | kg |
| Coated erythrosine | 1.75 | kg |

(corresponding to 1 kg of Erythrosine)

### Preparation of Finished Product

An automatic sachet filling machine is equipped with a paper/aluminum/polyethylene foil suitable to obtaining finished sachets of 14×10 cm. Each sachet is filled with 34.6 g of bulk product checking the net content of the sachets on a continuous basis. The content of such sachet, upon dissolution in 500 ml of water, gives an oral solution suitable for colon cleansing and staining as a preparation to chromoendocsopy of the colon. The amount of oral solution to be ingested to obtain a complete cleansing of the colon and contemporarily coloring the gut varies from 2 to 4 liters, from patient to patient, due to interindividual natural variability, on physiological conditions and on the food ingested.

### Example 6

### Sachet I for Preparation of Oral Solution

A composition of the powder mixture for the preparation or oral solution with methylene blue is prepared. The quantities in each sachet suitable to prepare 500 mL of solution are:

| | |
|---|---|
| Polyethyleneglycol 4000 | 28.32 g |
| Sodium sulfate | 2.729 g |
| Sodium bicarbonate | 0.809 |
| Sodium chloride | 0.704 |
| Potassium chloride | 0.356 |
| Acesulfame K | 0.0749 |
| Sodium cyclamate | 0.0922 |
| Saccarin acid | 0.0202 |
| Natural flavour | 0.315 |
| Coated methylene blue | quantity corresponding to 1 g of color |

### Example 7

### Sachet II for Preparation of Oral Solution

A composition of the powder mixture for the preparation or oral solution with methylene blue is prepared. The quantities in each sachet suitable to prepare 500 mL of solution are:

| | |
|---|---|
| Polyethyleneglycol 3350 | 28.32 g |
| Sodium sulfate | 2.729 g |
| Sodium bicarbonate | 0.809 |
| Sodium chloride | 0.704 |
| Potassium chloride | 0.356 |
| Acesulfame K | 0.0749 |
| Sodium cyclamate | 0.0922 |
| Saccarin acid | 0.0202 |
| Natural flavour | 0.315 |
| Coated methylene blue | quantity corresponding to 1 g of color |

### Example 8

### Mixture with Curcumin

Composition of the powder mixture for the preparation or oral solution with curcumin as coloring agent. The quantities in each sachet suitable to prepare 500 mL of solution are:

| | | |
|---|---|---|
| Polyethyleneglycol 4000 | 29.5 | g |
| Sodium sulfate | 2.843 | g |
| Sodium bicarbonate | 0.843 | g |
| Sodium chloride | 0.733 | g |
| Potassium chloride | 0.371 | g |
| Acesulfame K | 0.078 | g |
| Sodium cyclamate | 0.096 | g |
| Saccarin acid | 0.021 | g |
| Natural flavour | 0.315 | g |
| Curcumin | 4 | g |

(as enteric coated powder)

### Example 9

### Kit for 2L Solution

The quantities in each sachet or other container suitable to prepare 2 L of solution:

| | | |
|---|---|---|
| Polyethyleneglycol 3350 | 210 | g |
| Sodium bicarbonate | 2.86 | g |
| Sodium chloride | 5.6 | g |
| Potassium chloride | 0.74 | g |
| Enteric coated dye | 4 | g |

The kit may contain 1 or 2 tablets of laxative (biscodyl 5 mg).

### Example 10

### Mixture for Higher Osmolarity Solution

The quantities in each sachet or other container suitable to prepare 1000 mL of solution with an osmolarity of 392 mOsmol/kg are:

| | | |
|---|---|---|
| Polyethyleneglycol 3350 | 100.0 | g |
| Sodium sulfate | 7.5 | g |
| Absorbic acid | 4.7 | g |
| Sodium ascorbate | 5.9 | g |
| Sodium chloride | 2.69 | g |
| Potassium chloride | 0.93 | g |
| Flavor | 2.0 | g |
| Citric Acid | 1.565 | g |
| Saccarin acid | 0.021 | g |
| Natural flavour | 0.315 | g |
| Enteric coated dye | 1 | g |

(as enteric coated powder)

### REFERENCES

All publications and patents mentioned herein are hereby incorporated by reference in their entirety as if each individual publication or patent was specifically and individually incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

1. A dry composition comprising polyethylene glycol; an alkali metal sulfate;
an electrolyte selected from the group consisting of sodium bicarbonate, sodium chloride, and potassium chloride or a mixture thereof; and a gastro-protectant dye composition suitable for use in an internal colon examination procedure,
wherein the gastro-protectant composition comprises:
a gastro-protectant selected from the group consisting of methacrylic acid-methylmethacrylate copolymer and methacrylic acid-ethylacrylate copolymer and a dye.

2. The dry composition of claim 1, wherein the dye is microencapsulated by the gastroprotectant or the dye is film coated by the gastroprotectant.

3.-4. (canceled)

5. The dry composition of claim 1, wherein the gastro-protected dye composition comprises methylmethacrylate copolymer type A methacrylate.

6. The dry composition of claim 5, wherein the alkali metal sulfate is sodium sulfate.

7. (canceled)

8. The dry composition of claim 1, wherein the polyethylene glycol has an average molecular weight from about 3300 to about 4000 g/mol.

9. A container containing the dry composition of claim 1, wherein the container is a sachet, a packet, or an envelope.

10.-17. (canceled)

18. The dry composition of claim 1, further comprising ascorbic acid.

19. A colon cleansing and staining aqueous solution comprising the composition of claim 1, and about 2 to about 4 liters of water.

20.-21. (canceled)

22. A kit for a colonoscopy preparatory solution comprising:
a first container containing a first dry composition comprising:
25 g to 125 g of polyethylene glycol; and
0.5 g to 4 g of an alkali metal sulfate; and
0.3 g to 2 g of electrolytes selected from the group consisting of sodium bicarbonate, sodium chloride, and potassium chloride or a mixture thereof; and
a gastro-protected dye composition comprising a gastro-protectant selected from the group consisting of methacrylic acid-methylmethacrylate copolymer and methacrylic acid-ethylacrylate copolymer, a dye suitable for use in an internal colon examination procedure; and optionally lecithin.

23. (canceled)

24. The kit of claim 22, wherein the first dry composition comprises about 1 g to about 10 g gastro-protected dye composition.

25.-28. (canceled)

29. The kit of claim 22, any one of claims 22-28, comprising an additional container comprising ascorbic acid.

30. The kit of claim 22, further comprising a laxative.

31. The kit of claim 30, wherein the laxative is selected from the group consisting of bisacodyl, sodium picosulfate, and a senna-based laxative.

32.-41. (canceled)

42. The kit of claim 22, wherein the dye is methylene blue.

43. The dry composition of claim 1, wherein the dye is methylene blue.

44. The dry composition of claim 1, further comprising lecithin.
